(19) United States
(12) Patent Application Publication
(10) Pub. No.: US 2026/0294366 A1
(43) Pub. Date: Oct. 1, 2026

(54) METHOD FOR ACTUATING AN X-RAY IMAGING DEVICE

(71) Applicant: Siemens Healthineers AG, Forchheim (DE)

(72) Inventors: Matthias LICHY, Nuernberg (DE); Sebastian FABY, Forchheim (DE); Markus JUERGENS, Adelsdorf (DE)

(73) Assignee: Siemens Healthineers AG, Forchheim (DE)

(21) Appl. No.: 19/633,004

(22) Filed: Mar. 30, 2026

(30) Foreign Application Priority Data
Mar. 28, 2025 (EP) 25167064.2

(51) Int. Cl. A61B 6/42; A61B 6/00

(52) U.S. Cl. CPC A61B 6/4241; A61B 6/488; A61B 6/5223

(57) ABSTRACT

One or more example embodiments relates to a method for actuating an X-ray imaging device having at least one X-ray source and, opposite thereto, a photon-counting X-ray detector for acquiring a three-dimensional object image dataset of a mapping region of an object, the method comprising providing a first spectral image dataset that maps at least part of the mapping region of the object, deriving a setting of the X-ray imaging device based on the first spectral image dataset for the acquisition of the three-dimensional object image dataset, and actuating the X-ray imaging device to acquire the three-dimensional object image dataset based on the derived setting.

### CROSS-REFERENCE TO RELATED APPLICATION(S)

The present application claims priority under 35 U.S.C. § 119 to European Patent Application No. 25167064.2, filed Mar. 28, 2025, the entire contents of which is incorporated herein by reference.

### FIELD

One or more example embodiments relates to a method for actuating an X-ray imaging device having at least one X-ray source and, opposite thereto, a photon-counting X-ray detector for acquiring a three-dimensional object image dataset of a mapping region of an object based on a first spectral image dataset provided. Furthermore, one or more example embodiments relates to an actuation unit for actuating an X-ray imaging device, an X-ray imaging device and a computer program product.

Independent of the grammatical term usage, individuals with male, female or other gender identities are included within the term.

### RELATED ART

Modern imaging methods are often used to generate two or three-dimensional image data that can be used to visualize a mapped examination object and also for other applications. Imaging methods are often based on capturing X-ray radiation, wherein so-called projection measurement data, hereinafter also referred to as projection datasets, is generated. For example, projection measurement data can be acquired using a computed tomography device (CT device). In CT devices, a combination of an X-ray source and an opposite X-ray detector arranged on a gantry usually rotates around a measurement space in which the examination object is located. Herein, the center of rotation (also called the “isocenter”) coincides with a so-called system axis, which extends in the z-direction. During one or more rotations, the object is irradiated with X-ray radiation from the X-ray source, wherein the opposite X-ray detector is used to capture image datasets in the form of projection measurement data. These in each case comprise a plurality of projections for a plurality of angular directions containing information about the attenuation of the radiation by the examination object. Based on a set of projection measurement data, three-dimensional image datasets for a spatial representation of the object can then be generated via a suitable reconstruction algorithm.

In spectral computed tomography, as in other spectral imaging techniques, the different absorption levels of materials across an energy spectrum are used for further analysis. Herein, for example, in routine clinical practice, attenuation at low and high mean energies of the X-ray radiation emitted by the X-ray source is used for spectral CT imaging (“dual energy CT”). Herein, regardless of the type of data acquisition, the voltages at which the X-ray tube or tubes must emit in order to achieve sufficient spectral separation are generally specified in advance. The transmitted and attenuated X-ray quanta are thus split into two energy ranges, or the radiation required for image acquisition is divided in advance into a low and a high mean energy component and projection measurement data is acquired accordingly.

With the introduction of photon-counting detectors in computed tomography (photon counting CT, PCCT), the use of such photon-counting X-ray detectors also allows spectral information about the attenuation experienced or the energy of the detected X-ray quanta to be acquired independently of the emitted energy X-ray radiation output by the X-ray source, even at a (constant) tube voltage.

Herein, a suitable photon-counting X-ray detector can be used to separate the detected X-ray quanta into a plurality of energy classes depending on the energy thresholds set in the X-ray detector.

### SUMMARY

However, here once again, the energy of the thresholds required for this is generally defined in advance of image acquisition. This can, for example, result in insufficient data being obtained for further analysis for certain operations, such as iodine quantification, due to insufficient statistics in individual energy classes, or in the thresholds not being optimally set across the entire mapping region of the image dataset for later analysis. This can result in insufficient image quality and/or avoidably higher radiation exposure for a patient.

One or more example embodiments provides an improved method for actuating an X-ray imaging device, a corresponding actuation unit, an X-ray imaging device and a computer program product, which at least partially avoids the above-described disadvantages.

### BRIEF DESCRIPTION OF THE DRAWINGS

Exemplary embodiments of the invention are depicted in the drawings and are described in more detail below. The same reference symbols are used for the same features in different figures. The figures show:

FIG. 1 a schematic flowchart of a method for actuating an X-ray imaging device according to one or more example embodiments,

FIG. 2 a schematic representation of a mapping region on an object according to one or more example embodiments,

FIG. 3 a schematic representation of an arrangement of a photon-counting X-ray detector having a plurality of energy thresholds and an object according to one or more example embodiments,

FIG. 4 a schematic representation of an actuation unit according to one or more example embodiments, and

FIG. 5 a schematic representation of an X-ray imaging device according to one or more example embodiments.

### DETAILED DESCRIPTION

One or more example embodiments relates to a method for actuating an X-ray imaging device having at least one X-ray source and, opposite thereto, a photon-counting X-ray detector for acquiring a three-dimensional object image dataset of a mapping region of an object comprising the steps
providing a first spectral image dataset that maps at least part of the mapping region of the object,
deriving a setting of at least one actuation parameter of the X-ray imaging device based on the first spectral image dataset for the acquisition of the three-dimensional object image dataset,
actuating the X-ray imaging device to acquire the three-dimensional object image dataset based on the derived setting.

The X-ray imaging device is embodied to acquire a three-dimensional object image data set (3D object image dataset, hereinafter, also simply object image data set). The X-ray imaging device can in particular be embodied as a CT device. It can, for example, also be embodied as a C-arm X-ray device or another kind of X-ray device embodied to capture a 3D object image dataset.

A 3D image dataset allows three-dimensional representation, in particular spatially three-dimensional representation, of a mapping region. The 3D image dataset is generally based on a plurality of captured projection datasets via which the mapping region was scanned. Based on the captured projection datasets and a suitable reconstruction method, for example filtered backprojection, it is then possible to generate the spatially three-dimensional representation of the mapping region. A 3D image dataset can also be represented as a plurality of slice image datasets, so-called cross-sectional representations. A slice image dataset in each case comprises a slice of the 3D image dataset at a position along a designated axis. A slice image dataset in each case allows two-dimensional representation, in particular spatially two-dimensional representation, of the respective slice of the 3D image dataset.

Advantageously, such a 3D image dataset comprises a plurality of voxels, in particular image points. Herein, each voxel can preferably in each case have an image value, hereinafter also voxel value, in particular a CT image value in HU (“Hounsfield units”) in the case of a CT scan or an analogous intensity value. Analogously, a slice image dataset can comprise a plurality of pixels, in particular image points. Herein, each pixel can preferably in each case have an image value, in particular a CT image value in HU (“Hounsfield units”) in the case of a CT scan or an analogous intensity value.

A two-dimensional (2D) image dataset allows two-dimensional representation, in particular spatially two-dimensional representation, of a mapping region. A 2D-image dataset in particular has a plurality of pixels, in particular image points, wherein each pixel preferably in each case has an image value representing the locally detected intensity of the transmitted radiation.

The photon-counting X-ray detector used can in particular be a photon-counting direct-converting X-ray detector. In such X-ray detectors, incident X-ray radiation or photons can be converted into electrical pulses using a suitable converter material. Examples of converter materials that can be used are, for example, CdTe, CZT, HgI2, GaAs or others. The electrical pulses are evaluated by evaluation electronics, for example an integrated circuit (application specific integrated circuit, ASIC). In counting X-ray detectors, incident X-ray radiation is then measured by counting the electrical pulses produced by the absorption of X-ray photons in the converter material. The height or also the length of a generated electrical pulse is generally also proportional to the energy of the absorbed X-ray photon. This enables spectral information to be extracted by comparing the height or length of the electrical pulse with an energy threshold. Photon-counting X-ray detectors frequently have a plurality of settable energy thresholds for comparing the generated electrical pulses, thereby enabling energy-resolved measurements to be made as a function of a plurality of energy ranges defined by the energy thresholds.

In particular, the 3D object image dataset can be a spectral image dataset, in particular based on energy-resolved measurement data as a function of at least two energy thresholds.

The object can be a human and/or veterinary patient. However, it can also be an inanimate object, for example an examination phantom.

The provision of a first spectral image dataset, in particular a spectral X-ray image dataset, can in particular comprise capturing and/or reading a computer-readable data memory and/or receiving it from a data storage unit, for example a database. Furthermore, the image dataset can be provided by a processing unit of a medical imaging device for acquiring the spectral image dataset. The imaging device can, for example, comprise a CT device and/or a medical X-ray device, in particular a medical C-arm X-ray device, which is designed to measure spectrally resolved image datasets, in particular having a photon-counting X-ray detector. The imaging device can preferably be the X-ray imaging device to be actuated by the proposed method. However, it can also be another imaging device, in particular if the first spectral image dataset is based on a previously acquired spectral three-dimensional model dataset. The provision of a first spectral image dataset can also comprise generating, in particular reconstructing, the image dataset based on previously captured measurement data.

The first spectral image dataset can comprise a 2D-image dataset or a 3D image dataset. It can also be in the form of a projection dataset.

Preferably, the object is in an unchanged location and position when the data is captured for the first spectral image dataset and for the acquisition of the object image dataset, thereby enabling spatial transfer of the local properties of the object in a particularly advantageous and simple manner.

The first spectral image dataset can contain spectral information in the sense that the first spectral image dataset is based on measurement data representing the properties of the object with regard to the absorption of X-ray radiation by the object in a spectrally resolved manner, i.e., as a function of at least two mean energies or at least two energy ranges of the X-ray radiation. Preferably, the first spectral image dataset has been captured via a photon-counting X-ray detector and as a function of at least two energy thresholds. Based on this, a material property of the examination object, for example rather “water-like” or rather “iodine-like” can be inferred. For example, a material decomposition method can have been applied to the data underlying the first spectral image dataset. Based on the spectrally resolved first image dataset, it is possible to improve the prediction of the expected spectral absorption of X-ray radiation, in particular attenuation and/or beam hardening by the object, and/or expected statistics measured by the X-ray detector during X-ray fluoroscopy of the object during the acquisition of the object image dataset via the photon-counting X-ray detector, in particular also in a spatially resolved manner. The first spectral image dataset thus provides access to advantageously detailed spectral object information, which can be taken into account in the derivation step.

The first spectral image dataset provides spectrally resolved information for at least part of the mapping region. The first spectral image dataset can provide spectrally resolved information for the entire mapping region or even for the entire examination object.

Based on the first spectral image dataset, it is possible to derive an advantageous setting of the at least one actuation parameter which is used to actuate the X-ray imaging device to acquire the 3D object image dataset. The setting can be derived for the entire mapping region, for only part of the mapping region comprised by the first spectral image dataset or for a second part of the mapping region that is located close to the first part comprised by the first spectral image dataset. Accordingly, it can be expected that the properties of the object differ only insignificantly between this first part and this second part or can at least be derived therefrom. For example, the second part can be located directly adjacent to the first part. For example, the first spectral image dataset can comprise a spectral projection dataset captured during the acquisition of the three-dimensional object image dataset. For example, a setting can be derived for the acquisition of a subsequent projection dataset, for example the one directly following or the one following that, which maps a locally temporally subsequent subregion of the mapping region. If the derivation is performed for only part of the mapping region, repeated derivation for a further part can preferably take place within the framework of the method, so that, overall, the acquisition of the object image dataset results in a derived setting for the at least one actuation parameter for the entire mapping region. For example, derivation can be performed repeatedly based on spectral projection datasets captured during the acquisition of the three-dimensional object image dataset for subsequent projection datasets in each case.

The derived setting for the at least one actuation parameter can comprise a fixed value or also a value profile for the actuation parameter that is to be applied during the acquisition of the object image dataset and is taken into account accordingly when actuating the X-ray imaging device.

The actuation parameter can in particular comprise an actuation parameter of the X-ray tube or the X-ray detector. It can also comprise another actuation parameter. In particular, the at least one actuation parameter can comprise a parameter that has a direct influence on statistics measured in the X-ray detector and/or spectral separation and/or the measured spectral information. In particular, settings for a plurality of actuation parameters can also be derived.

According to one aspect of the proposed method, the at least one actuation parameter comprises at least one energy threshold of the photon-counting X-ray detector for the acquisition of the object image dataset. Advantageously, the energy thresholds of the X-ray detector can be optimally set based on the spectral information of the first spectral image dataset for the acquisition of the object image dataset and, for example, sufficient detected statistics can be ensured in each case as a function of the energy thresholds. Other parameters of the X-ray detector can also be comprised, for example signal amplification or pulse shaping time for the electrical signal generated in the detector in response to a detected X-ray photon.

The at least one actuation parameter can also comprise tube voltage, tube current or pre-filtering of the X-ray source and/or a rotation time or the relative displacement (“pitch”) between the object and the X-ray detector during the acquisition of the object image dataset. In addition, there may also be other parameters of the X-ray imaging device which can be derived within the framework of the proposed method. For example, the use of an additional spectrum can also be included as an actuation parameter.

The derivation of the setting of the at least one actuation parameter based on the first spectral image dataset enables the determination, in particular estimation, of expected spectral absorption during the acquisition of the object image dataset, in particular spectrally resolved attenuation and/or beam hardening by the object to be acquired. In particular, based on this, optimized setting of the at least one actuation parameter for the acquisition of the object image dataset, for example advantageously also in a spatially resolved manner across the mapping region, can be derived. Herein, in particular, expected measured statistics in the X-ray detector for the acquisition of the object image dataset, in particular also in a spatially resolved manner, across the mapping region can be better estimated, and, based on this, the setting of the at least one actuation parameter can be optimized. Optimized setting can in particular have the aim of ensuring that suitable measured statistics in the X-ray detector and/or image quality, for example quantified by a signal-to-noise ratio, are available in the object image dataset for later analysis and/or further processing of the object image dataset. Furthermore, the expected radiation exposure due to the acquisition of the object image dataset can be included. In addition to the object information provided by the first spectral image dataset, the clinical application underlying the object image dataset or the underlying reason for the imaging can also be included in the derivation. For derivation, simulated or measured models and/or historical image acquisitions on a patient population for the acquisition of the object image dataset can be included for the acquisition in respect to the at least one actuation parameter. Herein, it is also conceivable that an artificial intelligence method, for example a neural network, in particular a deep neural network, is used for derivation.

Based on the derived setting, the X-ray imaging device is then actuated for the acquisition of the object image dataset. For example, a control signal for the X-ray imaging device and/or individual components thereof can be provided to adapt the X-ray imaging device and/or the components thereof with respect to the actuation parameter according to the derived setting. Advantageously, an object image dataset, which has favorable image properties, in particular favorable image quality, can be acquired and then provided for analysis and/or further processing based thereon. This can in particular also enable unnecessary radiation exposure to be avoided.

In the framework of the method, a second or third spectral image dataset can also be provided based on which a derivation and thus an adaptation of the setting of the at least one actuation parameter is performed again, wherein the X-ray imaging device is then actuated based on the adapted setting. This can, for example, be used in perfusion imaging, wherein, before the acquisition of the actual object image dataset, a topogram can be repeatedly acquired in order to determine the optimal time for acquiring the measurement data. This can, for example, comprise repeatedly deriving the setting for subsequently captured projection datasets based on spectral projection datasets captured during the acquisition of the three-dimensional object image dataset.

According to one or more example embodiments, the first spectral image dataset can comprise a spectral topogram, a previously acquired spectral three-dimensional model dataset and/or a spectral projection dataset captured during the acquisition of the three-dimensional object image dataset.

Before the acquisition of projection data for a three-dimensional image dataset, in particular a CT image dataset, frequently an overview image data set, in most cases a two-dimensional overview image data set, of the examination object is generated, a so-called topogram. In routine clinical practice, the topogram does not contain spectral information. This can, for example, be used to define the acquisition region and thus the display region for the subsequent three-dimensional image dataset. Such a two-dimensional overview image data set can, for example, be generated in a CT device by continuously displacing the examination object laterally along the axis of rotation of the X-ray source while acquiring measurement data at fixed projection angles without rotating the X-ray source and the X-ray detector around the examination object during the acquisition. Based on this measurement data, it is then possible to assemble and display an overview image data set of the examination object. Particularly advantageously, a photon-counting X-ray detector can now be used to provide a spectral topogram with spectral information for the proposed method. This can furthermore advantageously provide spectral information across the entire mapping region, thereby enabling the setting of the at least one actuation parameter for the entire mapping region to be derived. Furthermore, a topogram is generally performed before a respective acquisition of the object image dataset so that no additional radiation exposure is necessary, wherein in particular a photon-counting X-ray detector can advantageously enable the spectral information to be obtained directly based on a plurality of energy thresholds without additional radiation exposure. Thus, it is, for example, possible to dispense with the use of two different tube spectra.

It is also possible to use a previously acquired spectral three-dimensional model dataset of the object for derivation. A model dataset can comprise a historical image dataset of the object. However, preferably, the model dataset is acquired in close temporal proximity to the object image dataset while maintaining the object's location and position, thereby ensuring sufficient consistency of the object properties. Three-dimensional information can allow an even more detailed derivation of the setting of the at least one actuation parameter.

It is also possible to use a spectral projection dataset captured during the acquisition of the three-dimensional object image dataset for derivation. This can enable advantageous derivation for subsequent projection datasets that is always adapted to the current conditions and measurement values and thus highly dynamic modulation during the acquisition of the object image dataset.

Furthermore, the first spectral image dataset can be based on energy-resolved measurement data based on at least three energy thresholds. Advantageously, high spectral resolution and thus detailed object information can be provided, thereby enabling improved derivation of an advantageous setting.

For example, more materials can be distinguished or the energy thresholds can advantageously be used in a targeted manner in order to assist the localization of special materials. For example, this would make it easier to localize implants in the object, for example gold fillings in teeth. It would also, for example, be conceivable to localize materials having a k-edge in a specific energy range, e.g., tracer materials or specially labeled drugs with k-edges in the range of 70-100 keV. In an advantageous variant, the first spectral image dataset is also based on the same number of energy thresholds as the object image dataset to be acquired, thereby enabling an advantageous setting of the energy thresholds to be derived particularly easily from the first spectral image dataset. Accordingly, fewer assumptions are required for the derivation.

According to one aspect of the proposed method, the derivation includes the clinical application of the imaging and/or the reason for the imaging underlying the object image dataset.

Advantageously, the expected objective and any boundary conditions of the imaging performed associated with the application of the imaging can be included in the derivation of the setting of the at least one actuation parameter and subsequently be optimized. For example, a thorax/upper abdomen scan, a lung scan or cerebral perfusion imaging is to be performed, each of which is associated with different objectives and conditions, for example the use of contrast agents, the actuation of the X-ray tube with regard to the emitted energy spectrum or the planned further processing of the image data with regard to a diagnosis based on the image data. For example, for the imaging, in particular also spectral imaging, of a specific target organ, the setting can comprise another value or value profile in regions away from the target organ, so that targeted analysis of the target organ is advantageously enabled, wherein away from the target organ, other values or another value profile of the actuation parameter lead to advantageous image results, enable another analysis or result in a lower required radiation exposure. Advantageously, not only object information but also application information is included in the derivation. Advantageously, statistics tailored to the object and the application can be achieved. This helps to avoid unnecessary radiation exposure and/or insufficient image quality.

As described above, the derived setting can comprise a fixed value for the at least one actuation parameter. The one value can be defined for the entire mapping region. For example, based on the information of the first spectral image dataset, a fixed set of energy thresholds for the X-ray detector can be defined for the entire mapping region to take into account the fact that suitable statistics are ensured throughout the mapping region as a function of the energy thresholds at the X-ray detector. This can also be applied to another actuation parameter in exactly the same way.

According to one or more example embodiments, however, the at least one actuation parameter can have different values during the acquisition of the three-dimensional object image dataset.

This can in particular comprise the at least one actuation parameter being modulated based on the information provided by the first spectral image dataset during the acquisition of the object image dataset. The setting of the at least one actuation parameter can thus advantageously be matched to the local circumstances in the mapping region. Advantageously, the properties of the object and, if applicable, the boundary conditions of the application of the imaging are taken into account in a spatially resolved manner, thereby ensuring that an advantageous object image dataset can be ensured. For example, based on the first spectral image dataset, a value profile for the at least one actuation parameter can be derived across the mapping region, based on which the X-ray device is actuated during the acquisition of the object image dataset. Accordingly, improved data acquisition, and thus improved image quality of the object image dataset, can be ensured in a spatially resolved manner.

According to one embodiment variant, the mapping region can be divided into at least two regions and the at least one actuation parameter can assume different values for the at least two regions. In particular, more than two regions can also be distinguished and the at least one actuation parameter can be set to more than two values.

The regions can be determined in advance in a particularly simple manner based on a spectral topogram or a spectral model dataset. In particular, regions along a longitudinal axis of the X-ray imaging device can be distinguished. The longitudinal axis can in particular be characterized by the fact that a system axis of the X-ray imaging device, in particular the axis of rotation of a CT device or a C-arm X-ray device for the acquisition of the object image dataset, runs parallel thereto. In particular, regions can be distinguished with different spectral absorption, so that adapted actuation parameters can lead to advantageous results. Furthermore, different regions can have different application objectives and thus a different optimized value for the actuation parameter. For example, the value for the at least one actuation parameter in a region with a high bone content differs from a region away therefrom. For example, for the imaging, in particular also spectral imaging, of a specific organ, a different value for the actuation parameter, for example different energy thresholds, can be advantageous for a region away therefrom. For example, for a target region for planned further processing of the image data with regard to the diagnosis based on the image data, a different value for the at least one actuation parameter is advantageous, whereas a different actuation parameter is selected away from this region.

Advantageously, spatially-resolved adaptation of the at least one actuation parameter can be ensured, while simultaneously achieving simple implementation. Herein, the spatial resolution of the modulation of the actuation parameter depends on the type and number of the selected regions.

In addition, it is also conceivable for a continuous value profile for the at least one actuation parameter to be derived as a function of the information provided by the first spectral image dataset, thereby achieving continuous adaptation of the actuation parameter during the acquisition of the object image dataset. For example, energy thresholds can be adapted continuously during the acquisition of the object image dataset.

According to one or more example embodiments, a plurality of projection image datasets can be captured for the acquisition of the three-dimensional object-image dataset, wherein the value of the at least one actuation parameter is adapted from projection dataset to projection dataset.

The adaptation can then comprise deriving an advantageous value for the at least one actuation parameter for each projection dataset. However, herein, adaptation can comprise the adapted value of the actuation parameter being equal to the value of the projection dataset provided that the same value was derived. This can in particular comprise the derivation being based in each case on a projection dataset captured during the acquisition of the object image dataset. Advantageously, this achieves high spatial and/or temporal resolution of the setting of the at least one actuation parameter as a function of the spectral information of the first spectral image dataset, thereby enabling highly dynamic modulation.

According to one or more example embodiments, the at least one actuation parameter can also have different values during the capture of a projection dataset comprised by the object image dataset.

For example, the at least one actuation parameter can also vary during the capture of a projection dataset. For example, depending on the relative position of the radiation source or the associated X-ray detector and of the object in the radial direction, optimized setting of the at least one actuation parameter can be achieved. For example, lateral fluoroscopy can be distinguished from frontal fluoroscopy of the object and suitable adaptation of the at least one actuation parameter can be achieved. Advantageously, high image quality can be achieved with potentially lower radiation exposure.

According to one or more example embodiments, the at least one actuation parameter comprises a parameter of the X-ray detector and the photon-counting X-ray detector comprises a matrix-like arrangement of a plurality of pixel elements, wherein the setting of the at least one actuation parameter for pixel elements of the plurality of pixel elements is at least partially different.

In particular, this can comprise being able to set the actuation parameter for the pixel elements of the plurality of pixel elements of the photon-counting X-ray detector spatially separately from one another and possibly accordingly also spatially separately adapt it during the acquisition of the object image dataset. For example, the actuation parameter comprises a number of settable energy thresholds for each pixel element of the plurality of pixel elements, wherein in the derivation step, different settings for the number of energy thresholds for the acquisition of the object image dataset are derived for different pixel elements of the plurality of pixel elements. In the actuation step, the pixel elements of the plurality of pixel elements can then be actuated differently based thereon, in accordance with the derived setting.

This can comprise in each case a derivation being available for each pixel individually, i.e., separately for each pixel element and the actuation parameter possibly also having different values for each pixel individually and/or being adapted for each pixel individually within the framework of the proposed method during the acquisition of the object image dataset. This can also comprise pixel elements being combined in groups, for example row-by-row, wherein a derivation is in each case performed for the group of pixel elements together and the actuation parameter may possibly also have different values group-by-group within the framework of the proposed method during the acquisition of the object image dataset and/or be adapted accordingly group-by-group. The groups can also be combined in other ways, for example pixel elements located in the center of the X-ray detector and pixel elements located at the edge, or in other ways.

This can in particular comprise varying settings for the at least one actuation parameter within the pixel matrix and, in addition, also a respective temporal modulation of the values during the acquisition of the object image dataset. For example, this can comprise continuously displacing a setting of the number of energy thresholds within the matrix of pixel elements during the acquisition of the object image dataset and in particular also as a function of the relative spatial position of the pixel element relative to the object in each case. Advantageously, the actuation parameter is set in a highly individual manner.

According to an advantageous embodiment, the setting of the at least one actuation parameter can vary along a longitudinal axis of the X-ray imaging device, in particular row-by-row or row-group-by-row-group, within the matrix-like arrangement of the plurality of pixel elements. This comprises in particular at least one row-by-row or row-group-by-row-group (i.e., in each case a plurality of rows) combination of the pixel elements of the pixel matrix with respect to the setting of the at least one actuation parameter. Within the row or row group, the same setting can then be derived for the pixel elements in each case, wherein it can differ accordingly row-by-row or row-group-by-row-group. This can represent an advantageous reduction in complexity compared to a pixel-individual derivation.

According to one or more example embodiments, the photon-counting X-ray detector has an extension of at least 6 cm along a longitudinal axis of the X-ray imaging device.

The large-area extension can enable rapid data acquisition across the mapping region. In particular, this can be particularly advantageous when used in conjunction with moving structures, for example to capture time-resolved image datasets. In the framework of the proposed method, this implementation is particularly advantageous in connection with the above-described aspect of a varying setting within the pixel matrix. In particular when using large-area X-ray detectors, in particular those with a larger extension along the longitudinal axis of the X-ray imaging device, a spatially separate setting within the pixel matrix is particularly advantageous for the acquisition of the object image dataset. During acquisition, such X-ray detectors in each case cover a region of the object in which significantly different object properties can already be present in different subregions. Spatially separate derivation and adjustability advantageously enable this to be taken into account and to ensure targeted and optimized setting of the at least one actuation parameter via the X-ray detector.

According to one or more example embodiments, the X-ray imaging device for the acquisition of the object image dataset can be operated in a sequential scan mode, wherein the at least one actuation parameter has different values at a first relative position between the object and the X-ray detector along a longitudinal axis of the X-ray imaging device and at a second relative position between the object and the X-ray detector along the longitudinal axis.

A sequential scan mode generally differs from spiral scanning in particular in that, during the capture of a projection dataset, i.e., for example during a rotation of the X-ray source/X-ray detector around the object, there is no simultaneous continuous relative movement between the object and the X-ray source/X-ray detector. Instead, relative movement takes place between the acquisitions of the projection datasets to capture a larger mapping region.

This aspect is in particular advantageous in conjunction with the above-described aspect of a larger extension of the X-ray detector along the longitudinal axis of the X-ray imaging device, since the larger coverage by the X-ray detector nevertheless enables rapid data acquisition. When combined with spatially resolved setting of the at least one actuation parameter within the pixel matrix of the X-ray detector, a sequential scan mode also, for example, allows simplified assignment and image reconstruction to be achieved despite the increased complexity of the measurement data, in contrast, for example, to combination with spiral scanning, since, in this case, there is no need to additionally take into account continuous displacement of the object.

Furthermore, in the framework of the proposed method, it is also conceivable that the information of the spectral image dataset could also be incorporated in processing steps downstream of the acquisition of the measurement data. For example, weighting of measurement data captured as a function of a plurality of energy thresholds can also be derived based on the first spectral image dataset.

One or more example embodimentsalso relates to an actuation unit for actuating an X-ray imaging device having at least one X-ray source and, opposite thereto, a photon-counting X-ray detector for acquiring a three-dimensional object-image dataset of a mapping region of an object, which is embodied to execute a method as described above as claimed in one of the preceding claims.

The advantages of the proposed actuation unit substantially correspond to the advantages of the proposed method for actuating an X-ray imaging device. Features, advantages or alternative embodiments mentioned here can also be transferred to the other claimed subject matter and vice versa.

The actuation unit can comprise a computing unit, a memory unit and/or an interface. It can also comprise a control unit. The actuation unit, in particular the components of the actuation unit, can be embodied to execute the individual steps of the proposed method and the above-described variants. The interface can be embodied to provide the first spectral image dataset. The computing unit and/or the memory unit can be embodied to derive a setting for at least one actuation parameter of the X-ray detector and/or the X-ray source based on the first spectral image dataset for the acquisition of the three-dimensional object image dataset. The control unit can be embodied to actuate the X-ray imaging device. The control unit can be embodied to provide a control signal to the X-ray imaging device based on the derived setting, so that the X-ray imaging device is operated in accordance with the derived setting during the acquisition of the object image dataset. The control unit can, for example, be embodied separately from the other components described, wherein a result of the derivation step can be output to the control unit via the interface, on the basis of which the control unit implements control of the X-ray imaging device.

The actuation unit can also comprise a user interface in the form of a display unit embodied to display the first spectral image dataset and/or the object image dataset or sectional representations thereof. This can be implemented in the form of a monitor, a touchscreen or another suitable display device. The actuation unit can also comprise a user interface in the form of an input unit embodied to enable manual user input. This can, for example, be enabled via a keyboard and mouse with corresponding input options or via another suitable input unit. Likewise, the input unit can be integrated in the display unit, for example in the form of a capacitive and/or resistive input display. Herein, the input unit can also enable control of the proposed method for actuating an X-ray imaging device via user input.

One or more example embodiments relates to an X-ray imaging device comprising an above-described actuation unit.

The advantages of the proposed X-ray imaging device substantially correspond to the advantages of the proposed method and/or the proposed actuation unit. Features, advantages or alternative embodiments mentioned here can also be transferred to the other claimed subject matter and vice versa.

The X-ray imaging device can preferably be embodied as a CT device. However, it can also comprise another medical X-ray device embodied to acquire a three-dimensional object image dataset, in particular for example a medical C-arm X-ray device.

In particular, the X-ray imaging device, in particular the CT device, advantageously comprises a photon-counting X-ray detector. In particular, a direct-converting X-ray detector can be embodied as a photon-counting X-ray detector, for example with CdTe, CdZnTe, CdTeSe, CdZnTeSe or CdMnTe or also another semiconductor material as the detection material.

One or more example embodiments also relates to a computer program product with a computer program, which can be loaded into a memory of an above-described actuation unit for actuating an X-ray imaging device, with program sections for executing all steps of the method for actuating an X-ray imaging device when the program sections are executed by the actuation unit.

The advantages of the proposed computer program product or computer-readable storage medium substantially correspond to the advantages of the proposed method. Features, advantages or alternative embodiments mentioned here can also be transferred to the other claimed subject matter and vice versa.

The computer program product can, for example, comprise software with a source code which still has to be compiled and linked or only has to be interpreted or an executable software code that only needs to be loaded into a corresponding computing unit for execution. The computer program product enables the method for actuating an X-ray imaging device to be executed quickly, identically repeatedly and robustly via an actuation unit. The computer program product is configured such that it can execute the method steps according to one or more example embodiments via the actuation unit.

The computer program product is for example stored on a computer-readable memory-medium or held resident on a network or server from where it can be loaded into the processor of an actuation unit which is directly connected to the actuation unit or can be embodied as part of the actuation unit. Furthermore, control information of the computer program product can be stored on an electronically readable data carrier. The control information of the electronically readable data carrier can also be embodied to perform a method according to one or more example embodiments when the data medium is used in an actuating unit. Examples of electronically readable data carriers are DVDs, magnetic tapes or USB sticks on which electronically readable control information, in particular software, is stored. When this control information is read from the data medium and stored in an actuation unit, all the embodiments of the above-described methods can be carried out.

A largely software-based implementation has the advantage that actuation units already in use can be easily retrofitted via a software update in order to operate in the manner according to one or more example embodiments. In addition to a computer program product, such a computer program product can also comprise additional items, such as, for example, documentation and/or additional components, and hardware components, such as for example, hardware keys (dongles, etc.) for using the software.

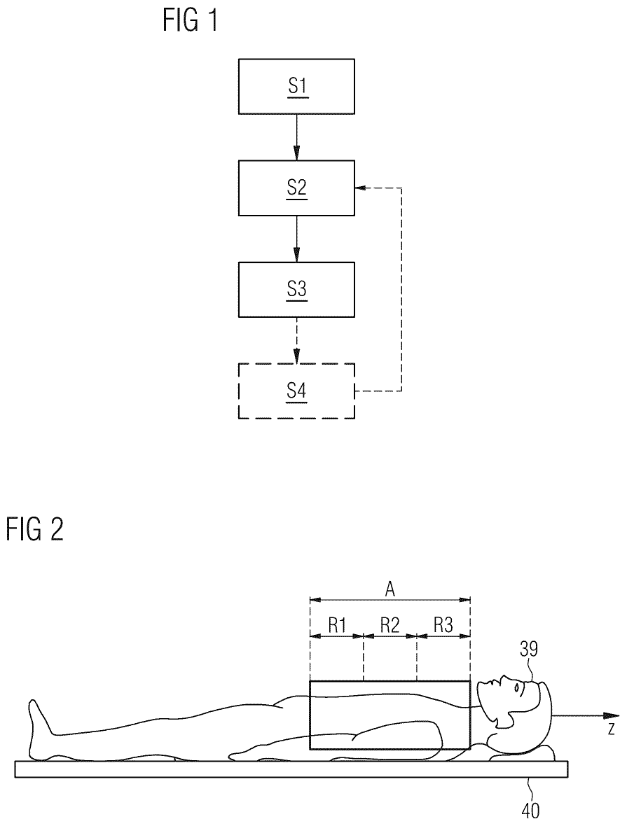

FIG. 1 shows a schematic flowchart of the proposed method for actuating an X-ray imaging device 32 having at least one X-ray source 37 and, opposite thereto, a photon-counting X-ray detector 36 for acquiring a three-dimensional object image dataset of a mapping region A of an object 39 comprising the steps
providing S1 a first spectral image dataset that maps at least part of the mapping region A of the object 39,
deriving S2 a setting of at least one actuation parameter T1, T2, T3 of the X-ray imaging device 32 based on the first spectral image dataset for the acquisition of the three-dimensional object image dataset,
actuating S3 the X-ray imaging device 32 to acquire the three-dimensional object image dataset based on the derived setting.

Furthermore, in some embodiment variants of the method, even after actuation S3, a second or third spectral image that maps, for example, a second part of the mapping region A of the object 39 or even the same part of the mapping region A at a different time can be provided S4 repeatedly and then made available for further derivation S2.

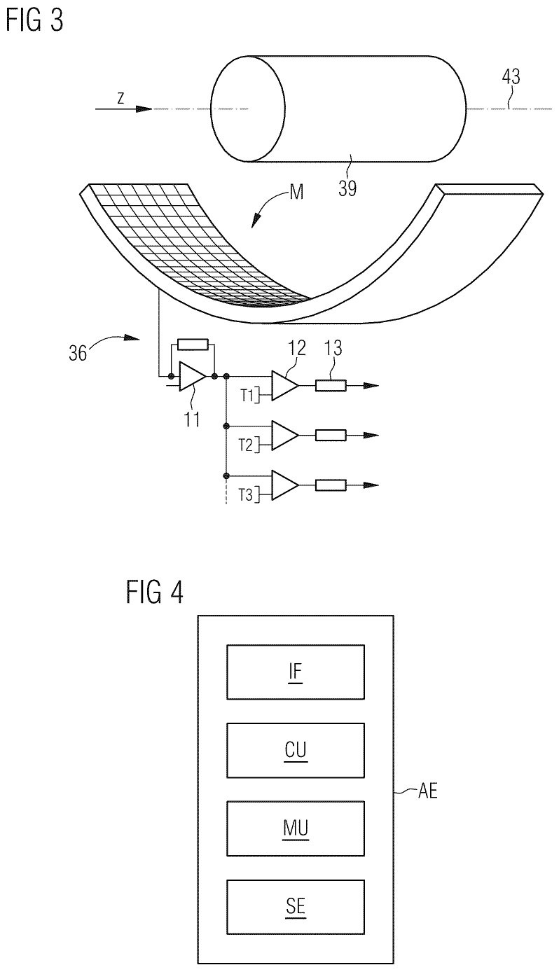

The X-ray imaging device 32 is for example embodied as a CT device or C-arm X-ray device. In particular, it is embodied to acquire a 3D image dataset that maps the mapping region A of the object 39. This can comprise capturing a plurality of projection datasets via which the mapping region is scanned. Herein, FIG. 2 shows by way of example a patient 39 on a patient positioning apparatus 40 of an X-ray imaging device, which is aligned along a longitudinal axis z of the X-ray imaging device 32. The mapping region A to be mapped via the object image dataset comprises a subarea of the patient 39, here in particular the thorax. Other mapping regions A can also be defined. In particular, the X-ray imaging device comprises a photon-counting X-ray detector, which is embodied to capture spectrally resolved measurement data based on at least two energy thresholds T1, T2, T3 (see also FIG. 3).

The first spectral image dataset can contain spectral information, at least for the part of the mapping region A comprised thereby, in the sense that the first spectral image dataset is based on measurement data representing the properties of the object 39 with regard to the absorption of X-ray radiation by the object 39 in a spectrally resolved manner, i.e., as a function of at least two mean energies or at least two energy ranges of the X-ray radiation. For example, the first spectral image dataset has been captured via a photon-counting X-ray detector 36 and as a function of at least two energy thresholds T1, T2, T3, in an advantageous embodiment of at least three energy thresholds. Based on the spectrally resolved first image dataset, it is possible to more accurately infer an expected spectral absorption of X-ray radiation, in particular attenuation and/or also beam hardening by the object 39 to be mapped, and/or expected statistics measured via the X-ray detector 36 during X-ray fluoroscopy of the object 39 during the acquisition of the object image dataset via the photon-counting X-ray detector 36, in particular also in a spatially resolved manner.

According to one or more example embodiments, the first spectral image dataset can comprise a spectral topogram, a previously acquired spectral three-dimensional model dataset and/or a spectral projection dataset captured during the acquisition of the three-dimensional object image dataset.

Based on the first spectral image dataset, a setting of the at least one actuation parameter is derived S2 and used to actuate the X-ray imaging device 32 for acquiring the 3D object image dataset. The setting can be derived for the entire mapping region A, only for the part of the mapping region A comprised by the first spectral image dataset or for a second part of the mapping region A that is located close to the first part comprised by the first spectral image dataset.

The derived setting for the at least one actuation parameter can comprise a fixed value or also a value profile for the actuation parameter that is to be applied during the acquisition of the object image dataset and is taken into account accordingly when actuating the X-ray imaging device 32. The actuation parameter can in particular comprise an actuation parameter of the X-ray tube 37 or the X-ray detector 36. In particular, the at least one actuation parameter can comprise a parameter that has a direct influence on statistics measured in the X-ray detector 36 and/or the spectral separation and/or the measured spectral information. In particular settings for a plurality of actuation parameters can also be derived.

In an advantageous embodiment, the at least one actuation parameter comprises at least one energy threshold T1, T2, T3 of the photon-counting X-ray detector 36 for the acquisition of the object image dataset. Alternatively or additionally, a setting of a tube voltage, tube current or pre-filtering of the X-ray source 37 and/or a rotation time or a relative displacement (“pitch”) between the object 39 and the X-ray detector 36 during the acquisition of the object image dataset can also be derived. In addition, there can also be further parameters of the X-ray imaging device 32. In particular, settings for a plurality of actuation parameters can also be derived.

The derivation of the setting of the at least one actuation parameter based on the first spectral image dataset enables the determination, in particular estimation, of expected spectral absorption during the acquisition of the object image dataset. In particular, based on this, an optimized setting of the at least one actuation parameter for the acquisition of the object image dataset, for example advantageously also in a spatially resolved manner across the mapping region, can also be derived. Herein, in particular expected measured statistics in the X-ray detector 36 for the acquisition of the object image dataset, in particular also in a spatially resolved manner across the mapping region, can be better estimated and, based on this, the setting of the at least one actuation parameter can be optimized.

For example, based on the first spectral image dataset, at least one energy threshold T1, T2, T3, preferably a plurality of energy thresholds T1, T2, T3, of the X-ray detector 36 is derived for the acquisition of the object image dataset, wherein it is more efficiently ensured that advantageous statistics are measured via the X-ray detector 36 across the mapping region as a function of the energy thresholds T1, T2, T3.

In addition to the object information provided by the first spectral image dataset, the clinical application underlying the object image dataset or the underlying reason for the imaging can also be included in the derivation. Advantageously, the expected objective and any boundary conditions of the imaging performed associated with the application of the imaging can be included in the derivation of the setting of the at least one actuation parameter and subsequently be optimized.

Based on the derived setting, the X-ray imaging device 32 is then actuated for the acquisition of the object image dataset. For example, a control signal for the X-ray imaging device 32 and/or individual components thereof can be provided in order to adapt the X-ray imaging device 32 and/or components thereof with respect to the actuation parameter according to the derived setting.

The derived setting can comprise a fixed value for the at least one actuation parameter. The value can be defined for the entire mapping region A. For example, based on the information of the first spectral image dataset, a fixed set of energy thresholds T1, T2, T3 for the X-ray detector 36 can be defined for the entire mapping region A, which takes into account the fact that suitable statistics are ensured throughout the mapping region A as a function of the energy thresholds T1, T2, T3 at the X-ray detector 36. This can also be applied to another actuation parameter in exactly the same way.

For example, a spectral topogram can be provided on the basis of which the expected spectral absorption, in particular attenuation and beam hardening, in the mapping region A can be determined in a spatially resolved manner. Then, a scan-based determination of the expected statistics as a function of the energy thresholds T1, T2, T3 and corresponding optimization of the energy thresholds T1, T2, T3 can be carried out. For example, models are used to predefine a fixed optimal threshold position for the entire mapping region A.

However, the at least one actuation parameter can also have different values during the acquisition of the three-dimensional object image dataset. This can in particular comprise modulating the at least one actuation parameter during the acquisition of the object image dataset, at least based on the information provided by the first spectral image dataset. As a result, the setting of the at least one actuation parameter can advantageously be adapted to the local circumstances in the mapping region A.

For example, the mapping region A can be divided into at least two regions R1, R2, R3 and the at least one actuation parameter can assume different values for the at least two regions R1, R2, R3. In particular, more than two regions R1, R2, R3 can also be distinguished and the at least one actuation parameter can be set to more than two values.

FIG. 2 shows by way of example a subdivision of the mapping region A into three regions R1, R2, R3, wherein different values for the at least one actuation parameter can be derived for the three regions R1, R2, R3.

In particular, regions R1, R2, R3 can be distinguished in which the spectral absorption differs, so that an adapted value for the actuation parameter leads to advantageous results. Furthermore, different regions R1, R2, R3 can have different application objectives and thus different optimized values for the actuation parameters. For example, for the imaging, in particular also spectral imaging, of a specific organ, a different value for the actuation parameter, for example other energy thresholds T1, T2, T3, can be advantageous compared to a region outside that organ.

In addition, it is also conceivable for a continuous value profile for the at least one actuation parameter to be derived as a function of the information provided via the first spectral image dataset, thereby achieving continuous adaptation of the actuation parameter during the acquisition of the object image dataset across the mapping region A. For example, energy thresholds T1, T2, T3 can be adapted continuously during the acquisition of the object image dataset.

It can also be provided within the framework of the method that a plurality of projection image datasets is captured for the acquisition of the three-dimensional object image dataset, wherein the value of the at least one actuation parameter is adapted from projection dataset to projection dataset. The adaptation can then comprise deriving an advantageous value for the at least one actuation parameter for each projection dataset, thereby achieving highly dynamic modulation.

It can also be provided that the at least one actuation parameter also has different values during the capture of a projection dataset comprised by the object image dataset.

According to one or more example embodiments, the at least one actuation parameter comprises a parameter of the X-ray detector 36 and the photon-counting X-ray detector 36 comprises a matrix-like arrangement M of a plurality of pixel elements, wherein the setting of the at least one actuation parameter for pixel elements of the plurality of pixel elements is at least partially different.

FIG. 3 shows by way of example a purely schematic representation of an arrangement of a photon-counting X-ray detector 36 having a plurality of energy thresholds T1, T2, T3 and an object 39 in an X-ray imaging device.

The arrangement is illustrated based on an arrangement of a CT device with an object 39 arranged along an axis of rotation 43 around which the X-ray detector 36 is rotatably arranged. The X-ray detector 36 has a matrix-like plurality of pixel elements thereby enabling spatially-resolved detection of X-ray radiation after passage through the object 39. Herein, the number of pixel elements depicted is by way of example only. The X-ray detector can in particular have a higher number of pixel elements than depicted here.

The X-ray detector is in particular embodied as a photon-counting direct-converting X-ray detector, wherein incident X-ray radiation or photons are converted by a suitable sensor converter material into electrical pulses. The converter material used can, for example, be CdTe, CZT, HgI2, GaAs or other materials. The electrical pulses are evaluated by evaluation electronics, for example an application specific integrated circuit (ASIC). Herein, by way of example, this is a schematic depiction of a processing chain connected to a pixel element of the sensor for the electrical pulses generated via the sensor in response to the detected photons generated in the evaluation electronics, which is generally present for each of the pixel elements, comprising a signal amplifier 11, a plurality of comparators 12 and counting elements 13 connected thereto in each case. The generated electrical signals are amplified and shaped if necessary via the signal amplifier 11 and then compared with a plurality of energy thresholds T1, T2, T3 via the comparators. If a signal exceeds an energy threshold T1, T2, T3, a counting event is generated and counted by the counting element 13. Accordingly, an incident X-ray spectrum can be measured with spectral resolution as a function of the set energy thresholds T1, T2, T3, on the basis of which an image dataset can be captured. By way of example, here, three energy thresholds are indicated, but there can also be more or fewer thresholds.

Particularly advantageously, the setting of the respective energy thresholds T1, T2, T3 of the X-ray detector 36 can be derived via the proposed method at least based on the first spectral image dataset and adapted for the acquisition of the object image dataset.

As described above, herein, the setting of the at least one actuation parameter for pixel elements of the plurality of pixel elements can be at least partially different. In particular, this can comprise it being possible to set the actuation parameter for the pixel elements of the plurality of pixel elements of the photon-counting X-ray detector 36 spatially separately and, if necessary, also to adapt it spatially separately during the acquisition of the object image dataset. For example, in the derivation step, different settings for the number of energy thresholds for the acquisition of the object image dataset are derived for different pixel elements of the plurality of pixel elements.

This can comprise a derivation being in each case available for individual pixels, i.e., separately for each pixel element and the actuation parameter may also have different values for each individual pixel and/or be adapted for each individual pixel within the framework of the proposed method during the acquisition of the object image dataset. According to an advantageous variant, this can also comprise pixel elements being combined in groups, in particular preferably row-by-row or row-group-by-row-group, wherein a derivation is performed in each case for the group of pixel elements and the actuation parameter possibly has different values for each group within the framework of the proposed method during the acquisition of the object image dataset and/or is adapted accordingly for each group.

This means that, for the at least one actuation parameter, a setting that varies within the pixel matrix M, in particular for example row-by-row or row-group-by-row group along a longitudinal axis z of the X-ray imaging device 32, and, in addition, a respective temporal modulation of the values can also take place during the acquisition of the object image dataset.

For example, a continuous row-by-row or row-group-by-row-group displacement of the energy threshold(s) T1, T2, T3 can be provided during the acquisition of the object image dataset, in particular as a function of a relative displacement between the X-ray detector 36 and the object 39.

For example, the mapping region A can be divided into a plurality of regions R1, R2, R3, wherein the energy threshold(s) T1, T2, T3 of the respective pixel element is/are adapted as a function of the relative position of a respective pixel element from the plurality of pixel elements to the regions. For example, a first part of the pixel matrix maps a first region, whereas a second part of the pixel matrix maps a second region and wherein the energy threshold(s) T1, T2, T3 of the first part of the pixel matrix has/have a different value adapted to the corresponding region R1, R2, R3 than the energy threshold(s) T1, T2, T3 of the second part. Herein, assignment to a first or second part of the pixel matrix can also change over time with a displacement between the object 39 and the X-ray detector 36.

The above-described spatial adaptability within the pixel matrix is particularly advantageous in combination with a large-area extension of the X-ray detector 36 along the z-direction, in particular with an extension of at least 6 cm. In particular, when using such large-area X-ray detectors 36, a spatially separate setting within the pixel matrix M is particularly advantageous for the acquisition of the object image dataset. During acquisition, such X-ray detectors in each case cover a region of the object 39 in which significantly different object properties can already be present in different subregions. Spatially separate derivation and setting advantageously enable this to be taken into account and to ensure targeted and optimized setting of the at least one actuation parameter via the X-ray detector 36.

Combination with the operation of the X-ray imaging device 32 in a sequential scan mode also represents a further advantageous variant, wherein the at least one actuation parameter has different values at a first relative position between the object 39 and the X-ray detector 36 along a longitudinal axis of the X-ray imaging device 32 and at a second relative position between the object 32 and the X-ray detector 36 along the longitudinal axis z. When combined with spatially resolved setting of the at least one actuation parameter, in particular the energy thresholds T1, T2, T3 within the pixel matrix M of the X-ray detector 36, a sequential scan mode can for example, enable simplified assignment and image reconstruction to be achieved despite the increased complexity of the measurement data, in contrast, for example, to combination with spiral scanning, since here it is not necessary also to take account of continuous displacement of the object 39.

Within the framework of the proposed method, a combination of the actuation of different actuation parameters is conceivable. For example, depending on the system and task, optimization of different parameters based on this combination could be provided, for example also flexible/dynamic modulation during the scan from projection to projection, based on the previous projection. For example, tube voltage adaptation over the shoulder region, an additional spectrum over a structure of interest in a dual source CT, possibly with different tube voltages, and an adaptation of pitch and rotation time for improved projections in the sense of better statistics for the respective task could be provided and, for example, combined with optimization of the energy thresholds.

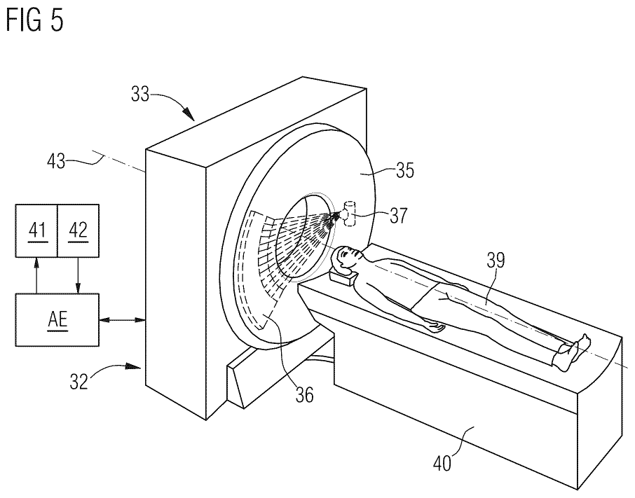

FIG. 4 is a schematic representation of an actuation unit AE, which is embodied to execute a proposed method and the aspects thereof. The provision unit AE comprises an interface IF, a computing unit CU and a memory unit MU. The actuation unit AE also comprises a control unit SE.

The components of the actuation unit AE can be connected to one another in order to enable efficient data exchange. The interface IF can be connected directly to the computing unit CU, which in turn can be connected to the memory unit MU. This arrangement can enable information to flow from the interface IF via the computing unit CU to the memory unit MU. The same applies to the control unit SE.

The actuation unit AE can be designed for data processing and storage. Herein, the interface IF can serve as an interface for input/output operations, the computing unit CU can perform calculation tasks and the memory unit MU can store data or results.

The interface can in particular be embodied to provide a first spectral image dataset. The computing unit CU can be embodied to execute the derivation steps of the proposed method and corresponding steps of embodiment variants thereof.

The memory unit MU can be embodied to store a first spectral image dataset. The memory unit MU can use different storage technologies to enable efficient management and rapid access to the stored data.

The control unit SE can be embodied to output a control signal for actuation of the X-ray imaging device 32 based on the derived setting.

FIG. 5 is a schematic representation of an X-ray imaging device 32.

In this example, the medical X-ray imaging device 32 is in particular a CT device comprising a radiation source, here in particular an X-ray source 37, a radiation detector, here in particular an X-ray detector 36, and an actuation unit AE. Herein, the X-ray source 37 and the X-ray detector 36 can be arranged opposite one another. The X-ray source 37 can be embodied to illuminate the X-ray detector 36 with X-ray radiation along an X-ray direction of incidence. The X-ray detector 36 is in particular a photon-counting direct-converting X-ray detector, for example with CdTe, CdZnTe, CdTeSe, CdZnTeSe or CdMnTe or also another semiconductor material as detection material.

The CT device 32 can comprise a gantry 33 with a rotor 35. The X-ray source 37 and the X-ray detector 36 can be arranged in a defined arrangement on the rotor 35, in particular integrated into the rotor 35 or attached to the rotor 35. The rotor 35 can be rotatably mounted about an axis of rotation 43. The longitudinal axis z of the X-ray imaging device 32 runs parallel to the axis of rotation 43. The CT device could also be embodied as a dual source device having two X-ray sources/X-ray detector combinations arranged offset from one another.

The examination object 39 to be mapped, here a human patient, is positioned on the patient positioning apparatus 40 and can be moved along the axis of rotation 43 through the gantry 32. The actuation unit AE can be used to control the CT device 32 and to calculate sectional images or volume images of the examination object 39. The actuation unit AE is in particular embodied to execute a method as proposed above for actuating an X-ray imaging device 32 and the aspects thereof.

The actuation unit AE is connected to an input unit 42, for example a keyboard and/or a mouse, and a display unit 41, for example a monitor and/or display. The input unit 42 can be integrated into the display unit 41, for example in the case of a capacitive and/or resistive input display. Herein, input by a medical operator at the input unit 42 can enable the actuation unit AE to be controlled. For this purpose, the input unit 42 can, for example, send a signal to the actuation unit AE. The display unit 41 can advantageously be embodied to display 2D and 3D image datasets and information and representations derived therefrom and, for example, sectional representations. For this purpose, the actuation unit AE can send a signal to the display unit 41. Furthermore, the input unit 42, and possibly the display unit 41, can enable manual input by a medical operator.

The schematic representations in the figures described do not represent any scale or proportions.

Finally, reference is made once again to the fact that the methods described in detail above and the apparatuses depicted are merely exemplary embodiments which can be modified in wide ranges without departing from the scope of the invention. Furthermore, the use of the indefinite article “a” or “an” does not preclude the possibility that the features in question could also be present on a multiple basis. Likewise, the terms “unit” and “element” do not preclude the possibility that the components in questions could consist of several interacting sub-components, which could also be spatially distributed.

In the context of the present application, the term “based on” can in particular be understood in the sense of the term “using”. In particular, wording according to which a first feature is generated (alternatively: ascertained, determined, etc.) does not preclude the possibility that the first feature could be generated (alternatively: ascertained, determined, etc.) based on a third feature.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for actuating an X-ray imaging device having at least one X-ray source and a photon-counting X-ray detector for acquiring a three-dimensional object image dataset of a mapping region of an object, the photon-counting X-ray detector opposing the at least one X-ray source, the method comprising:
providing a first spectral image dataset that maps at least a part of the mapping region of the object;
deriving a setting of at least one actuation parameter of the X-ray imaging device based on the first spectral image dataset for the acquisition of the three-dimensional object image dataset; and
actuating the X-ray imaging device to acquire the three-dimensional object image dataset based on the derived setting.

2. The method of claim 1, wherein the first spectral image dataset comprises at least one of a spectral topogram, a previously acquired spectral three-dimensional model dataset or a spectral projection dataset captured during the acquisition of the three-dimensional object image dataset.

3. The method of claim 1, wherein the first spectral image dataset is based on energy-resolved measurement data based on at least three energy thresholds.

4. The method of claim 1, wherein the deriving comprises determining an expected spectral absorption in the part of the mapping region comprised by the first spectral image dataset.

5. The method of claim 1, wherein the at least one actuation parameter comprises at least one of
an energy threshold of the photon-counting X-ray detector for the acquisition of the object image dataset,
a tube voltage, a tube current or a pre-filtering of the X-ray source, or
a rotation time or a relative displacement between the object and the X-ray detector during the acquisition of the object image dataset.

6. The method of claim 1, wherein the at least one actuation parameter has different values during the acquisition of the three-dimensional object image dataset.

7. The method of claim 6, wherein the mapping region is divided into at least two regions and the at least one actuation parameter has different values for the at least two regions.

8. The method of claim 6, wherein a plurality of projection datasets is captured for the acquisition of the three-dimensional object image dataset and wherein a value of the at least one actuation parameter is adapted from projection dataset to a next projection dataset of the plurality of projection datasets.

9. The method of claim 1, wherein the at least one actuation parameter has different values during a capture of a projection dataset comprised by the object image dataset.

10. The method of claim 1, wherein the X-ray imaging device is operated in a sequential scan mode and the at least one actuation parameter has different values at a first relative position between the object and the X-ray detector along a longitudinal axis of the X-ray imaging device and at a second relative position between the object and the X-ray detector along the longitudinal axis.

11. The method of claim 1, wherein
the photon-counting X-ray detector comprises a matrix-like arrangement of a plurality of pixel elements, the at least one actuation parameter if for pixel elements of the plurality of pixel elements, and
during the acquisition of the object image dataset, the setting of the at least one actuation parameter for the pixel elements of the plurality of pixel elements is at least partially different.

12. The method of claim 11, wherein the setting of the at least one actuation parameter varies along a longitudinal axis of the X-ray imaging device within the matrix-like arrangement of the plurality of pixel elements.

13. The method of claim 1, wherein the photon-counting X-ray detector has an extension of at least 6 cm along a longitudinal axis of the X-ray imaging device.

14. An actuation unit for actuating an X-ray imaging device having at least one X-ray source and a photon-counting X-ray detector for acquiring a three-dimensional object image dataset of a mapping region of an object, the photon-counting X-ray detector opposing the X-ray source, the actuation unit is configured to execute the method of claim 1.

15. An X-ray imaging device comprising the actuation unit of claim 14.

16. A non-transitory computer program product having instructions, when executed by an actuation unit, cause the actuation unit to perform the method of claim 1.

17. The method of claim 2, wherein the first spectral image dataset is based on energy-resolved measurement data based on at least three energy thresholds.

18. The method of claim 2, wherein the deriving comprises determining an expected spectral absorption in the part of the mapping region comprised by the first spectral image dataset.

19. The method of claim 2, wherein the at least one actuation
parameter comprises at least one of
an energy threshold of the photon-counting X-ray detector for the acquisition of the object image dataset,
a tube voltage, a tube current or a pre-filtering of the X-ray source, or
a rotation time or a relative displacement between the object and the X-ray detector during the acquisition of the object image dataset.

20. The method of claim 2, wherein the at least one actuation parameter has different values during the acquisition of the three-dimensional object image dataset.
